(19) United States
(12) Patent Application Publication
(10) Pub. No.: US 2004/0265953 A1
(43) Pub. Date: Dec. 30, 2004

(54) Production and use of inducible enzymes from trichoderma and bacteria for control of plant pests and for industrial processes

(76) Inventors: Gary E. Harman, Geneva, NY (US); Bruno Donzelli, Geneva, NY (US); Shiping Deng, Stillwater, OK (US)

Correspondence Address: Michael L. Goldman, Esq., NIXON PEABODY LLP, Clinton Square, P.O. Box 31051, Rochester, NY, 14603-1051, US

(21) Appl. No.: 09/927,984

(22) Filed: Aug. 10, 2001

Related U.S. Application Data
(60) Provisional application No. 60/225,700, filed on Aug. 11, 2000.

(51) Int. Cl. C12N 9/00; C12N 1/21

(52) U.S. Cl. 435/69.1; 435/252.3; 435/183

(57) ABSTRACT

The present invention relates to a method of increasing the expression of native extracellular inducible microbial enzymes from fungal sources using particular culture media and a method of repressing native proteins in fungi and enhancing the production of proteins encoded by transgenes in fungi under the control of an inducible promoter. The present invention also relates to mixtures of extracellular inducible enzymes from a fungal and a bacterial source as well as their use in a method for the degradation of chitinous substrates. Methods of releasing N-acetylglucosamine from chitinous sources and enhancing purity of a heterologous recombinant protein expressed in a culture of fungi are also disclosed.

The present invention claims benefit of U.S. Provisional Patent Application Ser. No. 60/225,700, filed Aug. 11, 2000.

This invention was developed with government funding by the US-Israel Binational Agriculture and Development (BARD) fund grant US-2325-93RC. The United States Government may have certain rights in the invention.

### FIELD OF THE INVENTION

The present invention relates to production and uses of extracellular inducible microbial enzymes from microbes for agricultural and nonagricultural uses.

### BACKGROUND OF THE INVENTION

Microorganisms in soil secrete a wide range of enzymatic proteins that degrade a wide range of substrates. These enzymes are broadly known as “extracellular” enzymes in reference to the fact that they are secreted from, and act outside of, the cell that produces them. Examples of substances degraded by microbial enzymes include, but are not limited to, cellulose, hemicellulose, chitin, protein, phytic acid and humic substances. In addition, they also have capabilities of degrading substances containing these compounds such as plant, animal, and microbial cells and tissues. Consequently, they regulate the accumulation of such compounds in soils and other environments.

Cellulose and hemicelluloses are together the most abundant polymers on earth. Enzymes that degrade such substances have wide industrial applications for fabric finishing, food processing, for improving quality of animal feeds, in the paper industry (Galante et al., “Application of Trichoderma Enzymes in the Textile Industry.” in Harman, G. E. and Kubicek, C. P. (ed.): Trichoderma and Gliocladium, Taylor and Francis, Vol. 2, London, pp. 291-309 (1998); Galante et al, “Applications of Trichoderma Enzymes in the Food and Feed Industries.” in Harman, G. E. and Kubicek, C. P. (ed.): Trichoderma and Gliocladium, Taylor and Francis, London, pp. 327-342 (1998); Buchert et al., “Applications of Trichoderma reesei Enzymes in the Pulp and Paper Industry.” in Harman, G. E. and Kubicek, C. P. (ed.): Trichoderma and Gliocladium, Vol. 2. Taylor and Francis, London, pp. 343-363 (1998)). In addition, complete degradation of lignocellulosic substrates is required for production of glucose and other simple sugars for the production of ethanol (Krishna et al., “Simultaneous Saccharification and Fermentation of Lignocellulosic Wastes to Ethanol Using a Thermotolerant Yeast.” Bioresource Technol. 77:193-196 (2001)).

Chitin is the second most abundant polymer. It is found in numerous organisms, including insects, fungi, the exoskeletons of insects and the shells of crustaceans such as lobsters and crabs. In all of these diverse organisms, chitin is a critical component of the outer walls or shells of these organisms. In fungi, chitin usually is associated with β-1,3 glucans or cellulose (primarily β-1,4 glucans). In addition, in insects, the peritrophic membrane contains chitin and lines the gut cavity. Disruption of fungal cell walls or of the peritrophic membrane by use of particular glucanolytic or chitinolytic enzymes results in control of the various fungi or insects, as demonstrated in U.S. Pat. Nos. 5,173,419, 5,326,561, and 5,433,947, all to Harman et al., and U.S. Pat. No. 6,069,299—Broadway et al.

Moreover, microbial strains that produce high levels of proteolytic enzymes were better for control of soil-inhabiting nematodes than strains that produced less of these enzymes (Sharon, et al., “Biological Control of the Root-Knot Nematode Meloidogyne javanica by Trichoderma harzianum,” Phytopathology 91:687-693 (2001)).

In some cases, pest control can be affected without direct damage to the pest; for example, enzyme mixtures have been prepared that have efficacy in removing nits from hair by dissolving the chitinous adhesive that holds the nits to hair.

Moreover, chitin and its products have a wide range of nonagricultural uses. For these uses, the native chitin must be modified to form various kinds of materials prepared by degradation of the native polymer to more useful forms. For example, chitin can be deacetylated to give rise to chitiosan, which is used as a neutriceutical used to absorb excess fats within the digestive system. Glucosamine is also a neutriceutical; it is formulated as a dietary supplement to improve joint health. Chitosan has antifungal activity and has been used, for example, to control post harvest diseases of strawberries (E1 Ghaouth et al., “Antifungal Activity of Chitosan on Two Postharvest Pathogens of Strawberry Fruits,” Phytopathology 82:398-402 (1992)). In addition, there are numerous potential uses of chitin derivatives in medicine, for example, to prepare flexible bandages. Further, it may be possible to degrade fungal cell walls or crustacean wastes, for example, to provide simple sugars utilizable for fermentation to ethanol or another energy source, as was described earlier for lignocellulosic materials. In addition, both N-acetylglucosamine and glucosamine can be used as pharmaceuticals to relieve joint stiffness and pain in humans and animals.

In most cases, these uses of enzymes require enzyme mixtures with different functions, typically enzymes that degrade polymers by an endo-type (random) activity, and exo-type activities that release small units from one end of the molecule. Typically, different classes of enzymes are synergistic in their activity. For example, endo- and exochitinases are more effective in controlling fungi than single enzymes (U.S. Pat. Nos. 5,474,926, 6,251,390, to Harman et al.). Degradation of lignocelluloses require potent mixtures of enzymes that are all required for degradation of the substrate (Koivula et al., “Structure-Function Relationships in Trichoderma Cellulolytic Enzymes,” in Harman, G. E. and Kubicek, C. P. (eds.): Trichoderma and Gliocladium, Vol. 2. Taylor and Francis, London, pp. 3-23 (1998)).

However, in some cases, production of single enzymes from microbial sources is useful. For example, individual enzymes may be added to an enzyme mixtures to optimize the mixture for a specific purposes, or may be required for the most efficient degradation of a substrate or precise modification of a substrate for a particular purpose.

Thus, for some purposes, specific individual enzymes must be produced, either from purification of complex mixtures or else by transgenic expression in systems that provide specific, nearly pure extracellular enzymes. In many other cases, natural or native enzymes offer useful methods to produce the enzymes.

There are numerous sources of useful enzymes. For example, Trichoderma spp. produce a wide range of chitinolytic enzymes that differentially cleave the chitin molecule (U.S. Pat. Nos. 5,474,926, 6,251,390, to Harman et al.; Lorito, “Chitinolytic Enzymes and Their Genes,” in Harman, G. E. and Kubicek, C. P. (eds.): Trichoderma and Gliocladium, Vol. 2. Taylor and Francis, London, pp. 73-99 (1998)). These same fungi also are efficient producers of enzymes that degrade celluloses, lignocelluloses, and hemicelluloses and proteins (Buchert et al., “Applications of Trichoderma reesei Enzymes in the Pulp and Paper Industry,” in Harman, G. E. and Kubicek, C. P. (eds.): Trichoderma and Gliocladium, Vol. 2. Taylor and Francis, London, pp. 343-363 (1998); Galante et al., R., “Application of Trichoderma Enzymes in the Textile Industry,” in Harman, G. E. and Kubicek, C. P. (eds.): Trichoderma and Gliocladium, Taylor and Francis, Vol. 2, London, pp. 291-309 (1998); Koivula et al, “Structure-Function Relationships in Trichoderma Cellulolytic Enzymes,” in Harman, G. E. and Kubicek, C. P. (eds.): Trichoderma and Gliocladium, Vol. 2. Taylor and Francis, London, pp. 3-23 (1998); Sharon et al., Biological Control of the Root-Knot Nematode Meloidogyne javanica by Trichoderma harzianum,” Phytopathology 91:687-693 (2001)). They also effectively degrade β-1,3 glucans (U.S. Pat. No. 5,474,926; Benitez et al., “Glucanolytic and Other Enzymes and Their Control,” in Harman, G. E. and Kubicek, C. P. (eds.) Trichoderma and Gliocladium, Vol 2. Taylor and Francis, London, pp. 101-127 (1998)).

A different group of extracellular enzymes are produced by Streptomyces spp. (Broadway et al., “Partial Characterization of Chitinolytic Enzymes from Streptomyces albidoflavus,” Lett. Appl. Microbiol., 20:271-276 (1995); U.S. Pat. No. 6,069,299 to Broadway et al.).

The present invention is directed to the improved production of extracellular microbial enzymes.

### SUMMARY OF THE INVENTION

The present invention relates to a method of increasing the expression of native extracellular inducible microbial enzymes from Trichoderma or Gliocladium species. This involves culturing Trichoderma or Gliocladium species in a medium containing material sufficient to induce expression of native extracellular inducible microbial enzymes, low levels of carbohydrates, and/or low levels of reduced nitrogen compounds under conditions effective to increase the expression of native fungal extracellular inducible microbial enzymes.

The present invention also relates to a method of repressing the expression of native proteins and enhancing the expression of proteins encoded by transgenes in a culture of fungi. This involves culturing fungi, which have been transformed with at least one transgene controlled by an inducible promoter, in a medium containing an inducer of the promoter, high levels of carbohydrates, and/or high levels of reduced nitrogen compounds, under conditions effective to repress expression of native proteins and enhance the expression of proteins encoded by the at least one transgene.

The present invention also relates to a composition containing a fungal source and a bacterial source of a extracellular inducible microbial enzyme.

The present invention also relates to a method of releasing N-acetylglucosamine from a chitinous source. This involves treating a chitinous source with a composition containing a fungal source of a extracellular inducible microbial enzyme and a bacterial source of a extracellular inducible microbial enzyme, under conditions which are effective to release N-acetylglucosamine from the chitinous source.

The present invention also relates to a method of repeatedly degrading different chitinous substrates. This is accomplished in a step-wise process involving (a) treating a first chitinous substrate with a composition containing a fungal source of a extracellular inducible microbial enzyme and/or a bacterial source of a extracellular inducible microbial enzyme under conditions effective to produce chitin. Next, in step (b), treatment step (a) is terminated. In step (c), a second chitinous substrate is treated with the same composition used to carry out step (a) under conditions effective to produce chitin.

The present invention also relates to a method of increasing expression of native extracellular inducible microbial enzymes from Trichoderma or Gliocladium species. This method involves culturing Trichoderma or Gliocladium species in a medium material sufficient to induce expression of native extracellular inducible microbial enzymes, a carbon source not repressive of expression of fungal extracellular inducible microbial enzymes, and/or low levels of reduced nitrogen compounds under conditions effective to increase the expression of native fungal extracellular inducible microbial enzymes.

The present invention also relates to a method of enhancing purity of a heterologous recombinant protein expressed in a culture of fungi. This method involves culturing the recombinant fungi in a medium containing nitrate as the medium's sole nitrogen source under conditions effective to induce expression of the heterologous protein while reducing expression of other proteins.

### BRIEF DESCRIPTION OF THE DRAWINGS

FIG. 1 is a schematic representation of the nag1, ech42, and gluc78 upstream regulatory sequences. The position of CRE1 (5′ SYGGRG 3′), GATA (5′ HGATAR 3′, 5′ GATT 3′), STRE (5′ CCCCT 3′), Br1A (5′ MRAGGGR 3′), AbaA (5′ CATTCY 3′) putative binding sites are reported. In the gluc78 URS, the gray bar represents an intron located in the 5′ non-coding region of the gene.

FIGS. 2A-C show Northern analyses of ech42, nag1, and gluc78 from T. atroviride after growth in enhanced minimal medium containing different levels of ammonium acetate (H=100 mM and L=10 mM) or glucose (H=3% and L=0.1%) and in the presence or absence of colloidal chitin (1.0%). Expression levels at 24, 48, and 72 hours after medium replacement are shown in FIGS. 2A, B, and C, respectively. All lanes were loaded with 7.5 μg of total RNA. Hybridizations were performed for 18 hours at high stringency using PCR-generated DIG-labeled dsDNA specific probes.

FIGS. 3A-C show expression, at 24, 48, and 72 hours after medium replacement, respectively, of nahase, endochitinase, and chitobiosidase by T. afroviride as assessed by separation of enzymes using native PAGE and visualization of activity from culture filtrates. Filtrates were harvested from cultures grown in enhanced minimal medium containing different levels of ammonium acetate (H=100 mM and L=10 mM) or glucose (H=3% and L=0.1%) and in the presence or absence of colloidal chitin (1.0%). Activity was assessed by release of methylumbelliferone by digestion of the appropriate methylumbelliferyl substrate. Culture filtrates (15 μl) were loaded for each lane and separated at constant voltage (4 V/cm).

FIG. 4 shows expression of β-1,3 glucosidase by T. atroviride as assessed by separation of enzymes using native PAGE and visualization of activity from culture filtrates. Filtrates were harvested from cultures grown in enhanced minimal medium containing different levels of ammonium acetate (H=100 mM and L=10 mM) or glucose (H=3% and L=0.1%) and in the presence or absence of colloidal chitin (0.2%). Activity was detected by a decrease in fluorescence of laminarin conjugates with aniline blue decolorized at pH 9.0. Culture filtrates (15 μl) were loaded for each lane and separated at constant voltage (4 V/cm).

FIGS. 5A-D show the differential induction of nahase, endochitinase, and chitobiosidase from T. atroviride by the addition of chitin or chito-oligomers to the culture media over time, as assessed by separation of enzymes using native PAGE and visualization of activity from culture filtrates. FIG. 5A shows the induction of a single nahase (CHIT773) after 4 hr culturing in media containing low levels of ammonium and high levels of glucose, with and without 1 mM of four oligomers of N-acetylglucosamine ((GlcNac)₁, (GlcNac)₂, (GlcNac)₃, or (GlcNac)₄). FIG. 5B shows induction of two forms of nahase (CHIT73 and CHIT42) after 12h in culture. FIG. 5C shows levels of both species of nahase increase after 24 hr in culture. FIG. 5D shows nahase induction after 36 hr in culture. Filtrates were harvested from cultures grown in enhanced minimal medium containing 10 mM of ammonium acetate and 3% glucose (H=3% and L=0.1%). Activity was assessed by release of methylumbelliferone by digestion of the appropriate methylumbelliferyl substrate. Abbreviations: 0=no chito-oligomers; 1=N-acetyl glucosamine; 2=N, N′ diacetylchitobiose; 3=N, N′ N″, triacetylchitotriose ((GlcNac)₃); 4=N, N′ N″, N′″, tetracetylchitotetrose ((GlcNac)₄); n=colloidal chitin. Culture filtrates (75 μ) were loaded for each lane and separated at constant voltage (4 V/cm).

FIGS. 6A-D show activity banding patterns and protein bands obtained from strain P1 and the transgenic strain VTT 95467 (“strain 67”) cultured in media formulations A-H, as indicated above each set of lanes. FIGS. 6A and 6B show enzyme activity stains, done as described for FIG. 5, and the lower two present total protein bands as revealed by silver staining. The arrows labeled “1” indicate the large molecular weight nahase; arrows labeled “2” indicate CHIT73; arrows labeled “3” indicate CHIT42, and those denoted as 4 indicate CHIT40 (chitobiosidase).

### DETAILED DESCRIPTION OF THE INVENTION

One aspect of the present invention relates to a method of increasing expression of native extracellular inducible microbial enzymes from Trichoderma or Gliocladium species. This involves culturing Trichoderma or Gliocladium species in a medium containing material sufficient to induce expression of native extracellular inducible microbial enzymes, with low levels of carbohydrates, and/or low levels of reduced nitrogen compounds. Reduced nitrogen compounds are used herein refers to compounds containing nitrogen that have undergone a reaction resulting in the addition of a hydrogen atom, or simply an addition of electrons.

One way of carrying out this aspect of the present invention is to culture Trichoderma or Gliocladium species in media containing low levels of ammonium as a reduced nitrogen source for efficient production of various fungal enzymes. Also suitable as a reduced nitrogen source are ammonium salts, glutamine, urea, amino acids and mixtures thereof, at comparable molar concentrations. In one embodiment of the invention, ammonium may be used at 10 mM, and may range from 3 to 40 mM. Any combination of these sources of reduced nitrogen are also suitable in the medium of the present invention. Low levels of ammonia in the culture media leads to a physiological state in the fungus hereafter described as nitrogen starvation. Under conditions of nitrogen starvation Trichoderma or Gliocladium species are induced to express a range of enzymes, including but not limited to proteinases, cellulases, hemicellulases, phtyases, and lipases. These include, for example, chitinolytic enzymes and glucanolytic enzymes, such as endochitinases, nahases, chitobiosidases, and 1-3-β-glucosidase. Such enzymes are hereinafter referred to as “extracellular inducible microbial enzymes,” (“EIMEs”), defined as proteins with catalytic abilities that are secreted into the medium in which a microorganism is growing, and that are induced and repressed by specific substances. Typically, secretion of inducible enzymes is repressed by the presence of glucose or other carbon catabolite repressors in the medium at concentrations described herein. EIMEs are typically produced under low concentrations of glucose or ammonia or other reduced nitrogen compounds and, at least under some conditions, secretion is enhanced by the presence of the substrate for the enzymes that act as inducers.

Expression of these extracellular inducible microbial enzymes frequently has been reported to be induced by fungal cell wall components and repressed by carbon catabolic repressors, such as glucose (Carsolio et al., “Characterization of ech-42, a Trichoderma harzianum Endochitinase Gene Expressed During Mycoparasitism,” Proc. Natl. Acad. Sci. USA 91:10903-109073 (1994); Garcia et al., “Cloning and Characterization of a Chitinase (CHIT42) cDNA from the Mycoparasitic Fungus Trichoderma harzianum,” Curr. Genet. 27:83-89 (1994); Peterbauer et al., “Molecular Cloning and Expression of the nag1Gene (N-acetyl-β-D-Glucosaminidase-Encoding Gene) from Trichoderma harzianum P1,” Curr. Genet. 30:325-331 (1996); Tronsmo et al., “Coproduction of Chitinolytic Enzymes and Biomass for Biological Control by Trichoderma harzianum on Media Containing Chitin,” Biol. Cont. 2:272-277 (1992), which are hereby incorporated by reference in their entirety). However, different studies have conflicting results; in some reports, starvation conditions were sufficient to trigger extracellular enzyme production (Limon et-al. “Primary Structure and Expression Pattern of the 33-kDa Chitinase Gene from the Mycoparasitic Fungus Trichoderma harzianum,” Curr. Genet. 28:478-483 (1995); Ramot et al., “Regulation of β-1,3-Glucanase by Carbon Starvation in the Mycoparasite Trichoderma harzianum.” Mycol. Res. 104:415-420 (2000), which are hereby incorporated by reference in their entirety) while data from other studies indicated that fungal cell walls or cell wall components were important or required for extracellular enzyme expression (de la Cruz et al., “A Novel Endo-β-1,3-Glucanase, BGN13.1, Involved in the Mycoparasitism of Trichoderma harziarnzm,” J. Bacteriol. 177:6937-6945 (1995); Garcia et al., “Cloning and Characterization of a Chitinase (CHIT42) cDNA from the Mycoparasitic Fungus Trichoderma harzianum,” Curr. Genet. 27:83-89 (1994), which are hereby incorporated by reference in their entirety). A transcription factor, Cre1 (Ilmen et al., “The Glucose Repressor Gene cre1 of Trichoderma Isolation and Expression of a Full-Length and a Truncated Mutant Form,” Mol. Gen. Genet. 251:451-460 (1996), which is hereby incorporated by reference in its entirety) was shown in vitro to bind to the upstream regulatory region of the gene encoding an endochitinase (ech42) from T. atroviride, suggesting that this factor may be involved in glucose repression in Trichoderma (Lorito et al., “Mycoparasitic Interaction Relieves Binding of the Cre1 Carbon Catabolite Repressor Protein to Promoter Sequences of the ech42 (Endochitinase-Encoding) Gene in Trichoderma harzianum,” Proc. Natl. Acad. Sci. USA 93:14868-14872 (1996), which is hereby incorporated by reference in its entirety).

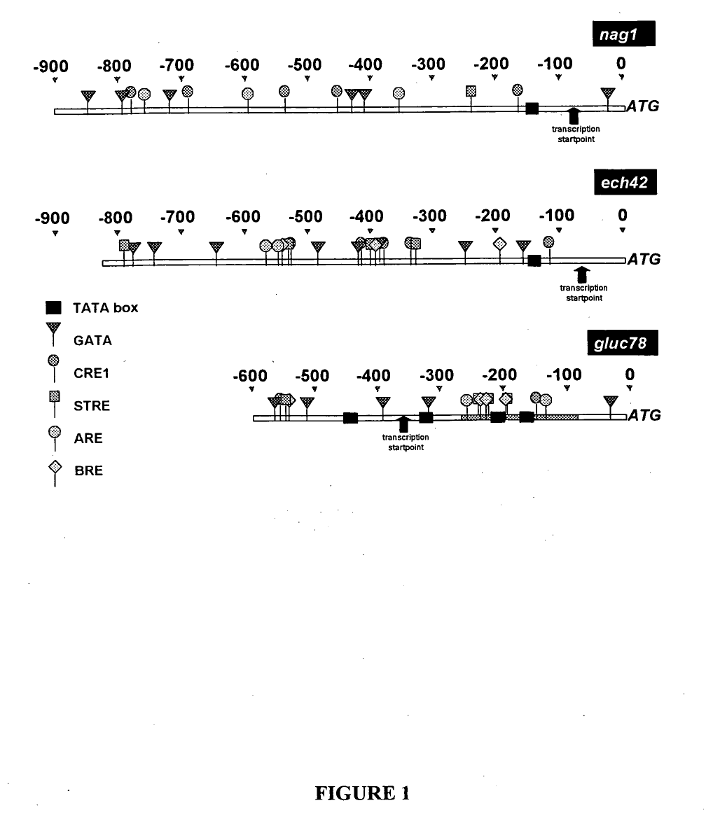

However, several lines of evidence indicate that extracellular enzymes, for example, cell wall degrading enzymes, are regulated by factors other than carbon catabolite repression and induction by fungal cell wall components. Carsolio et al., “Characterization of ech-42, a Trichoderma harzianum Endochitinase Gene Expressed During Mycoparasitism,” Proc. Natl. Acad. Sci. USA 91:10903-10907 (1994), which is hereby incorporated by reference in its entirety, reported the de-repression of ech42 in the presence of light. More recently, Mach et al., “Expression of Two Major Chitinase Genes of Trichoderma atroviride (T. harzianum P1) is Triggered by Different Regulatory Signals,” Appl. Environ. Microbiol. 65:1858-1863 1999), which is hereby incorporated by reference in its entirety, reported the inducing activity of chito-oligomers on the gene encoding a 73 kDa N-acetylhexosaminidase (nag1) but not on ech42 expression. Zeilinger et al., “Chitinase Gene Expression During Mycoparasitic Interaction of Trichoderma harzianum with its Host,” Fungal Genet. Biol. 26:131-140 (1999), which is hereby incorporated by reference in its entirety, showed that ech42 was induced prior to contact with a target fungus and suggested that this induction was mediated by chito-oligosaccharides released from the target fungus. nag1 was instead induced only after the Trichoderma-target fungus contact was established. Further, the upstream regulatory regions of gluc78 (encodes a glucan 1,3-β-glucosidase, hereafter designated 1,3-β-glucosidase), ech42 (encodes an endochitinase) and nag1 (encodes a N-acetylhexosaminidase) from T. atroviride contain clustered putative binding sites such as GATA, STREs, BREs and AREs, shown in FIG. 1, indicating the possibility of their regulation by a number of stimuli.

An alternative method of inducing nitrogen starvation is the substitution of nitrate for low levels of ammonia. Nitrate must be reduced in vivo to ammonium to be effective and this limiting step results in nitrogen starvation even when nitrate is used at higher concentrations. Nitrate may be used as a nitrogen source at concentrations ranging from 3 to 100 mM.

A further aspect of the invention is the use of glucose or other monosaccharides plus different levels of ammonia to achieve different purposes. If the goal is production of high levels of enzyme mixtures, then a combination of low levels of both glucose and ammonium may be used. This results in high and rapid expression of the enzymes noted above. Preferred levels of glucose or other monosaccharides are 0.1% w/v, while 0.05 to 0.5% may also be used. These levels of glucose or other monosaccharides are rapidly depleted and lead to a physiological state in the fungus hereafter described as carbon starvation. Carbohydrate sources suitable for use in the present invention include, but are not limited to, maltose, sucrose, galactose, mannitol, starch, hexoses, pentoses, sugar alcohols, disaccharides, amino sugars, easily degraded α-linked polymers, oligomers thereof, and mixtures thereof Also suitable are glucose, glucosamine, N-acetylglucosamine, galactose, galactosamine, N-acetylgalactosamine, fructose, mannose, ribose, arabinose, xylose, glucitol, mannitol and sorbitol.

The present invention also relates to increasing the expression of native EIMEs from Trichoderma or Gliocladium species by culturing Trichoderma or Gliocladium species in media with any chitin containing material as described above, and low levels of carbohydrates, and/or low levels of reduced nitrogen compounds in concentrations as previously described herein and further detailed in the Examples below.

Chitin or other complex carbohydrates such as those commonly found in plant or fungal cell walls also interact with glucose and nitrogen nutrition. Especially under conditions of nitrogen starvation and glucose sufficiency, these polymers are effective inducers of chitinolytic, but not glucanolytic, enzymes. In the preferred embodiment, chitin or other complex carbohydrates are used at concentrations of 0.05 to 1% w/v, and can be used at concentrations ranging from 0.02 to 5%. In addition to chitin, fungal cell walls, scleroglucans, crustacean shells and the like can be used as inducers of EIMEs. Further, if chito-oligomers ranging from N-acetylglucosamine to the tetramer (GlcNac₁₋₄, hereafter) are used under conditions of carbon sufficiency and nitrogen starvation, then specific nahases, together with endochitinase and chitobiosidase will be produced. Concentrations are as noted above for reduced nitrogen or other complex carbohydrates.

Another aspect of the present invention is a method of repressing the expression of native proteins and enhancing expression of proteins encoded by transgenes in a recombinant fungus. This method involves culturing fungi transformed with at least one transgene controlled by an inducible promoter, in a medium containing an inducer of the promoter, high levels of carbohydrates, and/or high levels of reduced nitrogen compounds, under conditions effective to repress expression of native proteins and to enhance expression of proteins encoded by the transgene. The promoter driving the gene of interest, irrespective of whether the gene is homologous or heterologous to the producing organism, will be insensitive to glucose, nitrogen and inducers noted above. In this case, highly efficient production of the enzyme can result from the producing organism being grown under carbon and nitrogen sufficiency. This will largely prevent expression of most of the other extracellular enzymes of the producing organism. Thus, this aspect of the present invention involves culturing a selected species of fungi with a high level of carbohydrate, ranging from 100 mM to 500 mM. In this aspect, nitrogen is also supplied to the media at a high concentration, in a range from 35 mM-500 mM. Any carbohydrate source and any reduced nitrogen source listed above is suitable for this aspect of the present invention. The medium will also contain a specific inducer of the promoter in question. Alternatively, the inducer can be added at a later stage of growth, which is a particularly useful technique if the product to be produced is toxic to the producing organism. These conditions can result in specific production of a single enzyme with only a small admixture of other proteins.

Methods for producing transgenic fungi and bacteria are well known (U.S. Pat. Nos. 5,474,926, 5,378,821, to Harmon et al.; Peterbauer et al., “Molecular Cloning and Expression of the nag1 (N-acetyl-β-D-Glucosaminidase-Encoding) Gene from Trichoderma harzianum P1,” Curr. Genet. 30:325-331 (1996); U.S. Pat. No. 6,069,299 to Broadway et al., which are hereby incorporated by reference in their entirety). In a typical transformation scheme, a fungus or bacterium is transformed with a DNA construct, in which a “foreign,” or heterologous, DNA molecule that is to be expressed is operably linked to a DNA promoter molecule that directs expression of the foreign DNA in the host cell, and to a 3′ regulatory region of DNA that will allow proper processing of the RNA transcribed from the target DNA. The choice of foreign DNA to be expressed will be based on the protein(s) one desires to produce. The promoter. molecule is selected so that the foreign DNA is expressed in the host. Promoters are regulatory sequences that determine the time and place of gene expression. Transcription of DNA is dependent upon the presence of a promoter which is a DNA sequence that directs the binding of RNA polymerase and thereby promotes mRNA synthesis.

Generally there are two types of promoters, constitutive and inducible. A constitutive promoter is a promoter that directs expression of a gene throughout the various parts of a plant and continuously throughout plant development. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide or phenolic compound, or a physiological stress directly imposed upon the host such as cold, heat, salt, or toxins. An exemplary promoter of the present invention is the cbh1 promoter, which is strongly responsive to cellulose as an inducer (Margolles-Clark et al. “Enhanced Expression of Endochitinase in Trichoderma harzianum Using the cbh1 Promoter of Trichoderma reesei,” Appl. Environ. Microbiol. 62:2152-2155 (1996), which is hereby incorporated by reference in its entirety).

The transgenic organism of the present invention is prepared having one or more heterologous DNA molecules, a suitable inducible promoter, and a 3′ regulatory region selected from among those which are capable of providing correct transcription termination and polyadenylation of mRNA for expression. The components are operably linked using methods generally known by those skilled in the art and as described by Margolles-Clark et al., “Enhanced Expression of Endochitinase in Trichoderma harzianum Using the cbh1 Promoter of Trichoderma reesei,” Appl. Environ. Microbiol. 62:2152-2155 (1996), which is hereby incorporated by reference in its entirety.

The transgenic organism of the present invention can also include a DNA molecule encoding a secretion signal. A number of suitable secretion signals are known in the art and others are continually being identified. The secretion signal can be a DNA leader which directs secretion of the subsequently translated protein or polypeptide, or the secretion signal can be an amino terminal peptide sequence that is recognized by a host secretory pathway. The secretion-signal encoding DNA molecule can be ligated between the promoter and the protein-encoding DNA molecule, using known molecular cloning techniques such as described in Sambrook et al., Molecular Cloning: A Laboratory Manual, Second Edition, Cold Spring Harbor Press, NY (1989), which is hereby incorporated by reference.

The transgenic organism is then cultured according to the present invention, with a suitable inducer and culture media selected to optimize heterologous protein production, as describe above and in Example 7, below.

The present invention also relates to a composition which contains a mixture of EIMEs from fungal and bacterial sources. Fungal sources suitable for this aspect of the present invention are, for example, filamentous fungi and yeast including, but not limited to, Trichoderma spp., Rhizopus spp., Aphanocladium spp., Coccidioides spp., Aspergillus spp., Hyprocrea spp., Candida spp., and mixtures thereof Exemplary bacterial sources for this aspect of the present invention include, but are not limited to, Aeromonas spp., Streptomyces spp., Serratia spp., Bacillus spp., Chromobacter spp., Vibrio spp., Pseudomonas spp., Pyrococcus spp., and mixtures thereof. Once produced, the enzymes of the present invention can be used for many purposes. The enzymes from Trichoderma are synergistic in their activities, for example, enzyme mixtures from Trichoderma are strongly synergistic in their ability to degrade fungal cell walls and control pathogenic fungi (U.S. Pat. No. 5,474,926 to Harman, which is hereby incorporated by reference in its entirety).

Fungi in the genus Trichoderma are found to be one of the most promising biocontrol agents against plant-pathogenic fungi (U.S. Pat. Nos. 5,165,928 to Smith; 5,173,419; to Harman et al.; G. E., “The Myths and Dogmas of Biocontrol. Changes in Perceptions Derived from Research on Trichoderma harzianum T-22,” Plant Dis. 84:373-393 (2000), which are hereby incorporated by reference in their entirety). The mechanisms by which they control plant-pathogenic fungi include antibiosis, competition, and mycoparasitism. Parasitism by Trichoderma spp. is destructive, causing death of the host fungus.

The complex process of mycoparasitism requires the production of enzymes that digest the fungal cell wall. Trichoderma spp. are known to be efficient producers of cell wall degrading enzymes, including polysaccharide lyases (for example, cellulases, chitinases, hemicellulases, α- and β-1,3 and 1,6 glucanases or glucosidases), proteases, phytases, and lipases.

There are three classes of chitinolytic enzymes produced by Trichoderma: N-acetyl-β-D-glucosaminidase (EC 3.2.1.30), chitiobiosidase, and endochitinase (EC 3.2.1.14) (Tronsmo et al., “Detection and Quantification of N-Acetyl-β-D-Glucosaminidase, Chitobiosidase and Endochitinase in Solutions and on Gels,” Anal. Biochem. 208 74-79 (1993), which is hereby incorporated by reference in its entirety). Purified proteins with all three types of activities from Trichoderma were strongly antifungal to different fungi (U.S. Pat. No. 5,474,926; to Harman et al.; Lorito et al., “Synergistic Interaction Between Cell Wall Degrading Enzymes and Membrane Affecting Compounds,” Molec. Plant-Microbe Interact. 9:206-213 (1996), which are hereby incorporated by reference in their entirety). Mixtures of different classes of chitinolytic enzymes exhibit synergistic effects in the inhibition of spore germination and hyphal elongation of plant pathogens in previous studies (U.S. Pat. No. 5,474,926, to Harman et al.; Lorito et al., “Potential of Genes and Gene Products from Trichoderma sp. and Gliocladium sp. for the Development of Biological Pesticides,” Molec. Biotechnol. 2:209-217 (1994), which are hereby incorporated by reference in their entirety). A number of genes encoding various enzymes from Trichoderma have been cloned and sequenced from Trichoderma (U.S. Pat. No. 5,378,821, to Harman et al.; Peterbauer et al., “Molecular Cloning and Expression of the nag1 (N-acetyl-β-D-Glucosaminidase-Encoding) Gene from Trichoderma harzianum P1,” Curr. Genet. 30:325-331 (1996), which are hereby incorporated by reference in their entirety). Genes with similar functions can also be cloned from other organisms such as bacteria in the genus Streptomyces (U.S. Pat. No. 6,069,299 to Broadway et al.). Genes encoding different proteins, even of the same general class, frequently are structurally unrelated to one another.

Chitinolytic enzymes may have been considered more widely for direct use in biocontrol than almost any other class of proteins. Enzymes within this group include many with different functions. Chitinases (EC 3.2.1.14) (hereafter described as endochitinases) cleave chitin chains randomly, exochitinases (N-acetylhexosaminidases, EC 3.2.1.52) (hereafter designated as nahases; they also have been named N-acetylglucosaminidases (nagases) or chitobiases and cleave a single glucosamine residue from the nonreducing end of the chitin chain and chitobiosidases cleave a dimer from the nonreducing end. Lysozymes (3.2.1.17) degrade bacterial cell walls and many also may be chitinolytic. All of these have been tested and many are in development for various purposes including production of disease- or insect-resistant transgenic plants or for production of enzymes for various biocontrol purposes from either engineered or native organisms. In addition, there are chitin deacetylases (3.2.1.41) that convert chitin to chitosan and there also are a series of chitosanases that are less well known. Chitinolytic enzymes are intrinsically interesting for biocontrol purposes because they degrade a polymer, chitin, which is present in pests such as fungi or insects but that is lacking in vertebrates or plants. Therefore, the supposition is that these enzymes would affect target species but have no effect upon humans or the crops they depend upon.

Chitinolytic enzymes are incredibly diverse and are produced by representatives of most phyla, including plants, vertebrates, fungi, eubacteria and archaebacteria. In nonchitin-containing plants and vertebrates, the enzymes are presumed to function as defensive agents against pest attack and are part of the induced resistance response of plants.

Moreover, mixtures of fungal source and bacterial source enzymes are more effective than either used alone in their abilities to degrade crustacean shells. Preferred ratios (volume/volume based on direct culture filtrates) are 75:25 fungal:bacterial enzymes although ratios ranging from 90:10 to 10:90 can also be used.

The present invention also relates to a method of releasing N-acetylglucosamine from a chitinous source. This method involves treating a chitinous source with an EIME from a fungal source and an EIME from a bacterial source under conditions effective to release N-acetylglucosamine, the monomeric amino sugar that is polymerized into chitin from the chitinous source. Suitable fungal sources of EIMEs include, but are not limited to, Trichoderma spp., Rhizopus spp., Aphanocladium spp., Coccidioides spp., Aspergillus spp., Hyprocrea spp., Candida spp., and mixtures thereof. Exemplary bacterial sources for this aspect of the present invention include, but are not limited to, Aeromonas spp., Streptomyces spp., Serratia spp., Bacillus spp., Chromobacter spp., Vibrio spp., Pseudomonas spp., Pyrococcus spp., Aeromonas spp., and mixtures thereof. This aspect of the present invention can be carried out under alkaline pH conditions to produce an oligomer of N-acetylglucosamine. A pH in the range of 8.0-11.0 provides suitably alkaline condition for this aspect of the present invention. Chitinolytic enzymes are useful for purposes other than control of plant pathogenic fungi. Insects and other arthropods, including shrimp, lobster, crabs and the like, also contain chitin in their exoskeleton. This insoluble polymer, and especially its degradation products, have many uses.

Another aspect of the present invention relates to a method of repeatedly degrading different chitinous substrates. This involves treating a chitinous substrate with a composition of the present invention containing a fungal source of a extracellular inducible microbial enzyme and/or a bacterial source of a extracellular inducible microbial enzyme under appropriate conditions. Suitable fungal sources of EIMEs include, but are not limited to, Trichoderma spp., Rhizopus spp., Aphanocladium spp., Coccidioides spp., Aspergillus spp., Hyprocrea spp., Candida spp., and mixtures thereof. Exemplary bacterial sources for this aspect of the present invention include, but are not limited to, Aeromonas spp., Streptomyces spp., Serratia spp., Bacillus spp., Chromobacter spp., Vibrio spp., Pseudomonas spp., Pyrococcus spp., and mixtures thereof. For example, langoustino shells are good inducers of chitinolytic enzymes from either the fungus T. atroviride or the bacterium Streptomyces albidoflavus in appropriate media. The enzyme mixture from T. atroviride at pH 5 provides release of glucosamine from langoustino shells, and so does the mixture from S. albidoflavus. At pH 9, however, the S. albidoflavus mixture releases only oligomers of chitin. Acidic reaction conditions range from pH 3.5-6.5; alkaline reaction conditions range from pH 8.0-11.0. Oligomers have different commercial uses than glucosamine. A 1:1 mixture of the bacterial and fungal enzymes at pH 5, for example, is synergistic in their release of glucosamine. Following the production of chitin from the first chitin containing substrate, the reaction is terminated. Termination of the reaction may be accomplished by physically separating the enzyme from the resultant degradation product, or the reaction can be self-terminated by allowing the reaction to continue until the initial substrate is exhausted. However, the enzymes of the composition are stable after 48 hr reaction times, so they can be reused, which dramatically improves their cost-effectiveness. Thus, this aspect of the present invention also involves treating a second chitinous substrate with the same composition used to carry out the first step. Reaction conditions are established as noted for the desired reaction. A third degradation reaction can also be carried out using the same enzyme composition as used for the previous two reactions. Each subsequent reaction can be initiated by adding fresh substrate directly to the previous reaction mixture, or by adding fresh substrate to the enzyme mixture removed and separated from the prior reaction step. The enzymes remain stable under the conditions of the present invention, and can be used repeatedly under optimized reaction conditions.

Moreover, enzyme mixtures from Trichoderma are strongly synergistic in their ability to degrade fungal cell walls and control pathogenic fungi (U.S. Pat. No. 5,474,926, to Harman et al., which is hereby incorporated by reference in its entirety).

Therefore, mixtures of the EIMEs of Trichoderma for this aspect of the present invention are also suitable for another aspect of the present invention is a method of increasing expression of native EIMEs from Trichoderma or Gliocladium species. This method involves culturing Trichoderma or Gliocladium species in a medium containing material sufficient to induce expression of native EIMEs, a carbon source not repressive of expression of fungal EIMEs, and/or low levels of reduced nitrogen compounds under conditions effective to increase expression of native fungal cell EIMEs. Non-repressive carbon compounds include those not involved in catabolite repression, and generally compounds not primarily involved in glycolysis or the pentose phosphate pathway, such as acetate. This aspect can be carried out with a source of chitin containing material sufficient to induce expression of native EIMEs. Sources of and concentrations for low levels of reduced nitrogen and sufficient chitin containing sources are as described above and in the Examples below.

Another aspect of the present invention relates to a method of enhancing purity of a heterologous recombinant protein expressed in a culture of fungi. This involves culturing the recombinant fungi harboring a transgene for a desired protein in a medium containing nitrate as the medium's sole nitrogen source under conditions effective to induce expression of the heterologous protein while reducing expression of other proteins. The recombinant protein is preferentially expressed while expression of native BIMEs are repressed. Therefore, the desired recombinant protein is relatively undiluted by other source proteins, and is easily recovered and purified.

Chitin, fungal cell walls and the cytoplasm of target fungi all are potential sources of both nitrogen and carbon for mycoparasitic Trichoderma spp. Therefore, it seems likely that Trichoderma spp. have evolved parasitic mechanisms to respond not only to the scarcity of simple carbon sources but to the lack of easily exploitable nitrogen compounds as well. This would require regulatory control of extracellular enzymes that respond to a lack of carbon or nitrogen, or both, in order to yield these nutrients from target fungi.

### EXAMPLES

### Example 1

### Nitrogen Starvation Triggers Microbial Inducible Extracellular Enzyme Production

Trichoderma atroviride (formerly designated T. harzianum) strain P1 (ATCC 74058) was used in these experiments. Conidia were collected from colonies sporulating on potato dextrose agar (Difco, Detroit, Mich.) and used to inoculate 250 ml Erlenmeyer flasks containing 50 ml potato dextrose broth (Difco, Detroit, Mich.) to give 2—10⁷ spores/flask. Cultures were incubated at 25° C. on a rotary shaker (150 rpm) for 18-24 hr. The germlings were collected on Miracloth (Calbiochem, La Jolla, Calif.), washed 3 times with sterile deionized water and used to inoculate 250 ml Erlenmeyer flasks containing 50 ml of enhanced minimal medium (EMM) that contained per liter: 23.4 g 2-[N-morpholino]ethanesulfonic acid, 5 g KH₂PO₄, 0.6 g MgSO₄, 0.6 g CaCl₂, 5 mg FeSO₄7H₂O, 10 mg FeCl ₃, 1.6 mg MnSO₄ H₂O, 1 mg ZnSO₄7H₂O, 1 mg CoCl₂, 40 μg CuSO₄5H₂O, and 13 μg (NH₄)₆Mo₇O₂₄4H₂O. Glucose was the carbon source and the nitrogen source was ammonium acetate. In preliminary experiments, ammonium sulfate was compared to ammonium acetate with similar results. Colloidal chitin (2 g/L dry weight, w/v) or dried mycelia of Botrytis cinerea (0.2 g/L dry weight, w/v) were added in some cases. Media were adjusted to pH 6.0. This system resulted in a uniform biomass at the start of each experiment and obviated such issues as poor germination of conidia in some media. To avoid browning of the media that occurred during autoclaving, EMM salt base, glucose and ammonium salt stock solutions were autoclaved separately and then blended under aseptic conditions.

### Example 2

### Enzyme Activity Studies

Chitinolytic activity was measured in the presence of 50 mM sodium acetate buffer at pH 5.0. For N-acetylhexosaminidase, the substrate was 4-methylumbelliferyl N-acetyl-β-D-glucosaminide (MUA) and for endochitinase the substrate was 4-methylumbelliferyl β-D-N,N′-diacetyl-chitobioside (MUB), both at 0.1 mM final concentration. Release of 4-methylumbelliferone after 30 min at 25° C. was measured using a fluorescence (360 nm excitation, 460 nm emission) microtiter plate reader (Cytofluor II; PerSeptive Biosystems, Framingham, Mass.).

Activity of β-1,3 glucanase was quantified using laminarin (Sigma, St. Louis, Mo.) as the substrate with a modification of the fluorometric method described by Cotéet al., “Detection of β-1,3-Glucanase Activity After Native Polyacrylamide Gel Electrophoresis: Application to Tobacco Pathogenesis-Related Proteins,” Electrophoresis 10:527-427 (1989), which is hereby incorporated by reference in its entirety). Laminarin was used at 0.1% in 12.5 mM sodium acetate, pH 5.0, final concentrations. After incubation for 30 min at 37° C., samples were stained with decolorized aniline blue and the reduction in fluorescence over time was measured by using Cytofluor II.

Nitrogen starvation was shown to trigger chitinase production. The media used (EMM) contained glucose at repressive levels (3% w/v), dried mycelia of B. cinerea (0.02% w/v) as an inducer, and nitrogen either as ammonium sulfate (5 or 50 mM) or ammonium acetate (10 or 100 mM). Both N-acetylhexosaminidase and endochitinase activities were high in culture filtrates when ammonium was present at 10 mM, but low when media contained 100 mM of ammonium. Similar results were obtained with either ammonium sulfate or ammonium acetate, but pH was more stable in media containing ammonium acetate and all subsequent experiments used ammonium acetate as the nitrogen source.

Expression of ech42, nag1, and gluc78 was affected by both carbon and nitrogen availability. Experiments were designed to examine the interaction of nitrogen, glucose, and the presence of colloidal chitin on expression of three genes that encode extracellular enzymes: ech42 (U.S. Pat. 5,378,821 to Harman et al., which is hereby incorporated by reference in its entirety), nag] (Peterbauer et al., “Molecular Cloning and Expression of the nagl (N-acetyl-B-D-Glucosaminidase-Encoding) Gene from Trichoderma harzianum P1,” Curr. Genet. 30:325-331 (1996), which is hereby incorporated by reference in its entirety), and gluc78.

### Example 3

### mRNA Transcription Analysis

RNA was extracted from about 100 mg of mycelia (Chomczynski et al., “Single-Step Method of RNA Isolation by Acid Guanidinium Thiocyanate-Phenol-Chloroform Extraction,” Anal. Biochem. 162:156-159 (1987), which is hereby incorporated by reference in its entirety). Total RNA was separated electrophoretically on a 1.2% formaldehyde-agarose gel and transferred onto a MagnaGraph nylon membrane (MSI, Westboro, Mass.). Probes prepared from the coding regions of ech42, nag1, and gluc78(850, 1400, and 1100 nucleotides, respectively) were labeled with PCR DIG Probe Synthesis Kit following the manufacturer's protocols (Roche Molecular Biochemicals, Indianapolis, Ind.). Membrane pre-hybridization, hybridization development, and stripping were performed as described by Roche protocols. CDP-STAR (Molecular Biochemicals, Indianapolis, Ind.) was used as the chemiluminescent substrate.

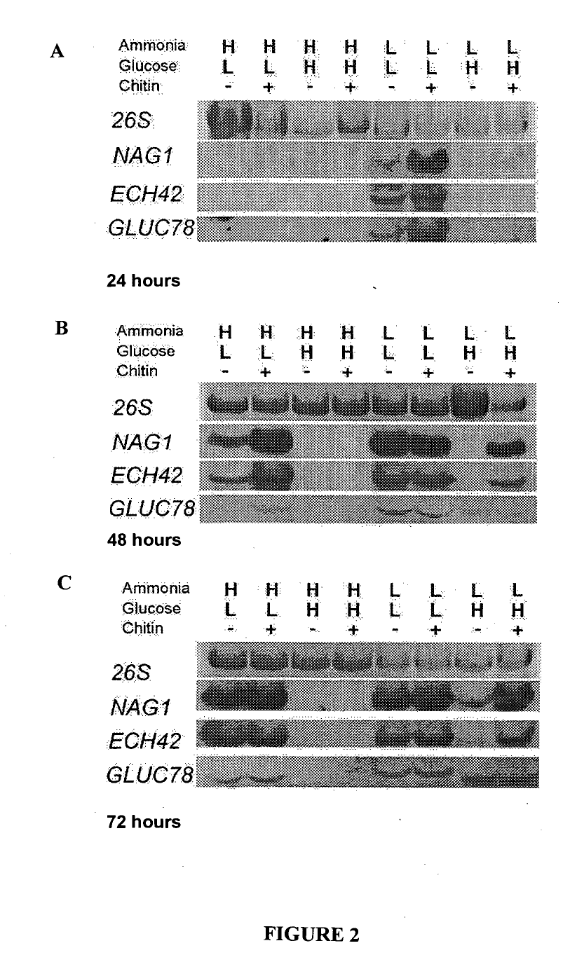

Northern analysis showed that in low ammonium and high glucose media, mRNA transcription of ech42 and nag1 occurred within 48 hr in the presence of high levels of glucose, shown in FIG. 2, when chitin was present in the medium. When chitin was absent, mRNA transcription was evident only 72 hr after the induction. Under these conditions, low gluc78 expression levels were detected 48 hr after the induction. Expression of this gene was unaffected by chitin, as shown in FIG. 2.

When glucose was present at low concentrations (0.1%) in the presence of high levels of ammonium (100 mM), nag1 and ech42 transcription occurred within 48 hr, regardless of the presence or absence of chitin as an inducer, shown in FIG. 2. However, when chitin was present, initial expression levels were higher, while 24 hr later no differences were evident. Expression of gluc78 occurred within 48 hr in the presence of chitin but only after 72 hr of culture in the absence of the polysaccharide.

When both carbon and nitrogen were at low levels, i.e., in low ammonium and low glucose containing media, nag1, ech42, and gluc78 were all expressed at high levels within 24 hr, and the presence of chitin had little additional effect. These results are shown in FIG. 2.

None of the three genes were expressed when both ammonium and glucose were present at high concentrations, either in the presence or absence of chitin.

### Example 4

### PAGE Analysis of Enzyme Activity.

Polyacrylamide gel electrophoresis (PAGE) of culture filtrates was carried out under native conditions (Harris et al., “Protein Purification Methods: A Practical Approach,” New York:IRL Press, which is hereby incorporated by reference in its entirety). Chitinase activity gels were performed using MUA and MUB as the substrates (Tronsmo et al., “Detection and Quantification of N-acetyl-β-D-Glucosaminidase, Chitobiosidase and Endochitinase in Solution and on Gels,” Anal. Biochem. 208:74-79 (1993), which is hereby incorporated by reference in its entirety). Activity of β-1,3 glucanase were examined on activity gels were performed using laminarin as the substrate (Cotéet al., “Detection of β-1,3-Glucanase Activity after Native Polyacrylamide Gel Electrophoresis: Application to Tobacco Pathogenesis-Related Proteins,” Electrophoresis 10:527-427 (1989), which is hereby incorporated by reference in its entirety).

In the presence of low ammonium, high glucose and chitin, activities of both endochitinase (CHIT42) and 73 kDa N-acetylhexosaminidase (CHIT73) were present in the culture filtrate by 48 hr. By 72 hr a band corresponding to a 40 kDa chitobiosidase (Harman et al., “Chitinolytic Enzymes of Trichoderma harzianum: Purification of Chitobiosidase and Endochitinase,” Phytopathology 83: 313-318 (1993), which is hereby incorporated by reference in its entirety).

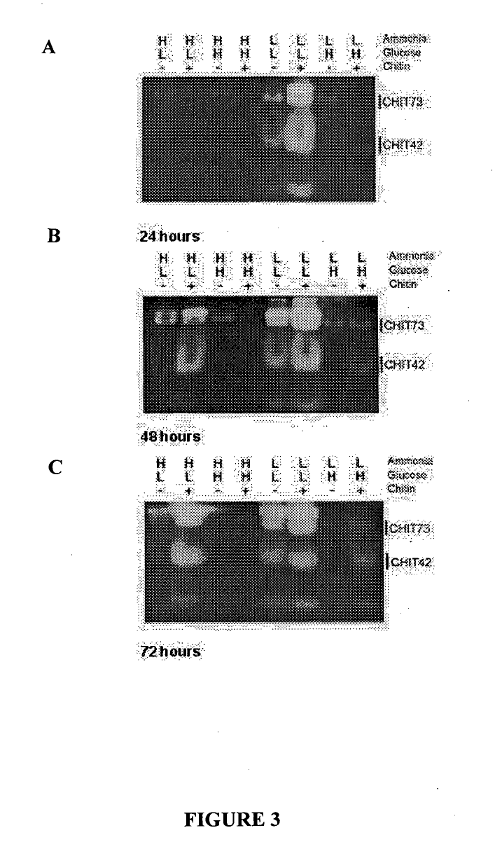

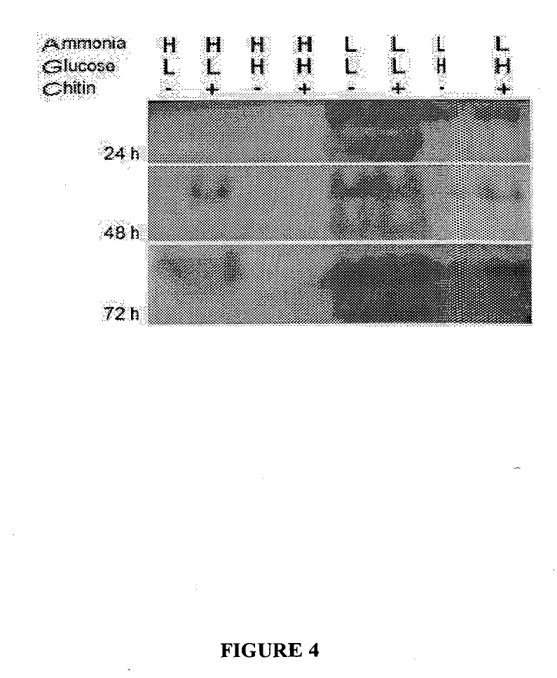

CHIT40 was also evident, as shown in FIG. 3. In the absence of chitin, CHIT42 was not detected. Low constitutive levels of nahase were detected at all times, shown in FIG. 3. Shown in FIG. 4, GLUC78, or a band that migrates to the same gel location, was present at all times tested with and without chitin. Activity was detectable at 24 hr, but transcripts were not. The enzyme increased in intensity between 48 hr and 72 hr, as seen in FIG. 4.

In high ammonium and low glucose containing media, nahase and endochitinase activities were detectable 48 hr and 72 hr after induction, with higher activity levels in the presence than in the absence of chitin, as shown in FIG. 3. At 72 hr CHIT40 also was present in filtrates from chitin-supplemented cultures (Harman et al., “Chitinolytic Enzymes of Trichoderma harzianum: Purification of Chitobiosidase and Endochitinase,” Phytopathology 83: 313-318 (1993), which is hereby incorporated by reference in its entirety). Two nahase bands were evident; the one with the greatest mobility corresponded to CHIT73, shown in FIG. 3. As show in FIG. 4, activity of β-1,3 glucanase was evident in chitin-containing cultures after 48 hr. After 72 hr, this activity was strong in both chitin-supplemented and chitin-free cultures.

Activities of nahase, chitinase, chitobiosidase, as shown in FIG. 3, and glucanase, shown in FIG. 4, were strong in cultures containing low ammonium and low glucose at all times, in both the presence and absence of chitin. In addition to CHIT73 and GLUC78, other bands with nahase and β-1,3 glucanase activity were detected, as shown in FIGS. 3 and 4, respectively. This was the only condition under which an additional β-1,3 glucanase band was evident, as shown in FIG. 4.

In cultures containing high glucose and high ammonium, neither chitinolytic nor glucanolytic enzyme activities were evident, as shown in FIG. 3 and FIG. 4, except for an extremely low level of nahase. This enzyme was detected at 24 and 48 hr in the absence, but not in the presence, of chitin.

Chitinolytic and glucanolytic enzymes play crucial roles in biocontrol of fungi by Trichoderma spp. Important to understanding their ecological role are factors affecting regulation of these genes.

High levels of ammonia repress expression of a glucanolytic and two chitinolytic enzymes, and also reduce levels of other extracellular chitinolytic and glucanolytic enzymes secreted by T. atroviride into culture media. These results are consistent with a recent report that ammonia represses another chitinolytic gene, chit33, in a strain of T. harzianum (de las Mercedes Dana, “Regulation of Chitinase 33 (chit33) Gene Expression in Trichoderma harzianum,” Curr. Genet. 38:335-342 (2001), which is hereby incorporated by reference in its entirety).

### Example 5

### Interactive Effects of Glucose and Ammonia on Expression of EIMEs

It was also discovered that glucose and ammonia have interactive effects on expression of chitinolytic and glucanolytic genes and enzymes:(a) complete repression resulted when both nutrients were present at high levels; (b) they were strongly expressed when both carbon and nitrogen were at low levels.

The level of glucose and ammonia also affected whether or not chitin induced expression of chitinolytic enzymes. When ammonium was at low levels and glucose was at high levels, enzyme expression occurred only in the presence of chitin or chito-oligomers. Thus, under this condition, chitin or chito-oligomers were essential for ech42 and nag1 transcription. Conversely, when either low glucose and high ammonium or low carbon and low nitrogen were present in the medium, chitin was not essential but enhanced and hastened expression of nag1, ech42, and gluc78.

These results help explain some of the discrepancies between different studies on the abilities of chitin to induce extracellular enzymes; Mach et al. “Expression of Two Major Chitinase Genes of Trichoderma atroviride (T. harzianum P1) is Triggered by Different Regulatory Signals,” Appl. Environ. Microbiol. 65:1858-1863 (1999), which is hereby incorporated by reference in its entirety, reported that that chitin or chito-oligomers were unable to induce transcription. Instead, ech42 was expressed, with the same strain used in this study, only after prolonged carbon starvation or stresses such as ethanol or cold shocks. However, they used a medium that contained high levels of nitrogen (urea and ammonia), so their results would be analogous to our high ammonia conditions (repressive to ech42). This comparison indicates the need to consider multiple repressive and inductive components of media or environment when studying expression of extracellular inducible microbial enzymes. Moreover, transcription of gluc78 was controlled differently from chitinolytic enzymes, since it was affected by the presence of chitin only under carbon starvation.

In some cases, there were apparent discrepancies between patterns of gene expression and enzymes present in the medium. For example, under conditions of carbon starvation (in the absence of chitin) and under nitrogen starvation (in the presence of chitin), transcription levels of ech42 and nag1 were comparable to the carbon plus nitrogen starvation. However, lower levels of CHIT42 (encoded by ech42) and CHIT73 (encoded by nag1) were detected in culture filtrates relative to the double starvation condition. Similarly, while nag1 transcription was strongly induced by the combination of nitrogen starvation and chitin, the amount of the protein detected in the medium was low. These results point to the presence of control at the post-transcriptional level for both nag1 and ech42 gene products.

Under some circumstances, including repressive conditions, low levels of nahase activity were detected even though no transcription of nag1 occurred. The nahase produced under this condition has lower electrophoretic mobility than CHIT73, suggesting that this protein is likely to be encoded by a gene other than nag1.

No doubt control of enzyme expression as noted in this study is of ecological importance. For example, in soils the level of available organic carbon and nitrogen sources and inorganic nitrogen are below the lowest levels used in this study (Marschner, Mineral Nutrition of Higher Plants, 2nd ed. Academic Press Ltd., San Diego, Calif. (1995), which is hereby incorporated by reference in its entirety). Even in the rhizosphere where root exudates increase the amount of easily exploitable compounds such as simple sugars and organic acids, the amount of readily available nitrogen (ammonia salts, amino acids) is likely to be much lower than 10 mM. In other words, soil conditions appear favorable for the expression of Trichoderma extracellular inducible microbial enzymes, even in relatively nutrient rich niches such as, rhizodeposition sheaths (Marschner, Mineral Nutrition of Higher Plants, 2nd ed. Academic Press Ltd., San Diego, Calif. (1995), which is hereby incorporated by reference in its entirety).

### Example 6

### Differential Induction of Microbial EIMEs

Enzyme mixtures were produced from T. atroviride in the EMM basic medium described in Example 1 but that contained 1 mM of four oligomers of N-acetylglucosamine ((GlcNac)₁, (GlcNac)₂, (GlcNac)₃, or (GlcNac)₄) (all from Sigma Chemical Co, St. Louis, Mo.). The media also contained low levels of ammonium and high levels of glucose (10 mM of ammonium acetate and 3% glucose, respectively). One hr after adding biomass to EMM, the oligomers were added separately to give a final concentration of 1 mM each. Chitin or no oligomers were added to separate media as controls.

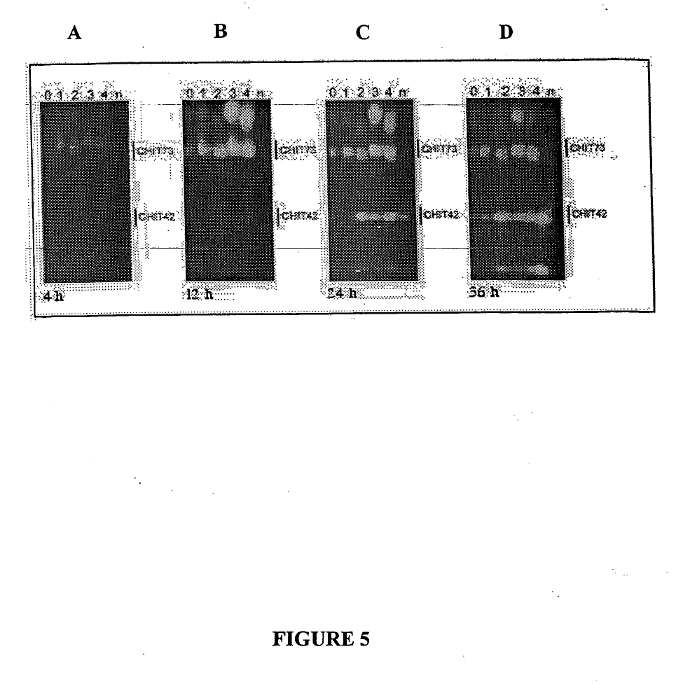

After 4 hr of culturing in the presence of chito-oligomers, single nahases were produced when (GlcNac)₁ to (GlcNac)₄ were present individually in the medium, as shown in FIG. 5A. After 12 hr, shown in FIG. 5B, several different forms of nahases were detected in the cultures without chitin with the greatest levels in media containing (GlcNac)₃ and (GlcNac)₄. At 24 and 36 hr., shown in FIGS. 5C and 5D, respectively, the induction pattern of the nahases were unchanged. By 36 hr, activity levels of nahases with low electrophoretic mobilities decreased relative to enzymes at or near the mobility level of CHIT73, FIG. 4. CHIT42 activity was detectable, at low levels, at 12 hr in cultures containing (GlcNac)₃ and (GlcNac)₄ FIG. 4. At 24 hr, CHIT42 activity was strong in the media supplemented with (GlcNac)₂₋₄ while lower levels of both CHIT42 and CHIT40 (a 40 kDa chitobiosidase) were detectable in chitin-supplemented media. Very low levels of these two enzymes were present in media supplemented with N-acetylglucosamine or that lacked chitin derivatives. At 36 hr CHIT42 was detectable in all the cultures, with the strongest activity in media supplemented with chitin.

This demonstrates that chitinolytic enzymes are differentially induced by different chito-oligomers under conditions of low ammonium and high glucose concentrations (the condition requiring chitin or chito-oligomers for chitinolytic enzyme production). GlcNac₁₋₄ were efficient inducers of nahases, but specifically induced particular enzymes; this specific induction has not heretofore been described. Chito-oligomers of different chain lengths induce different nahases and chito-oligomers are more effective in inducing production of CHIT42 and CHIT40 than colloidal chitin.

### Example 7

### Nitrogen Starvation Induced By Nitrate Enhances Heterologous Enzyme Production.

T. atroviride strain P1 was used in this example; in addition, transgenic strain VTT 95467 was prepared from strain P1. It carries an extra copy of ech42 driven by the cellobiohydrase promoter cbh1 of T. reesei to provide over-expression of the gene. It was prepared as described by Margolles-Clark et al. “Enhanced Expression of Endochitinase in Trichoderma harzianum Using the cbh1 Promoter of Trichoderma reesei,” Appl. Environ. Microbiol. 62:2152-2155 (1996), which is hereby incorporated by reference in its entirety. The cbh1 promoter is strongly responsive to cellulose as an inducer.

The strains were grown in eight different media in an effort to identify an enzyme mixture with a high level of antifungal activity. These media, designated A, B, C, D, E, F, G, and H, vary in mineral nutrients, trace elements, or carbon source.

Medium A contains 10 g of KNO3, 5 g of KH2PO4, 1.3 g of MgSO4, 20 mg of FeCl3, 3.5 mg of ZnSO4.7H2O, 0.4 mg of CuSO4.5H2O, 0.31 mg of MnSO4, 0.13 mg of (NH4)6Mo7O24.4H2O, 0.5 g sucrose, and 10 g crab shell chitin (Sigma) in 1 liter of water.

Medium B contains the same composition of mineral nutrients as in medium A, but 10 g of cellulose (Sigma) was used instead of chitin.

Medium C contains 5 g of lactose, 1 g of glucose, 5 g of (NH₄)₂SO4, 15 g of KH₂PO₄, 0.6 g of MgSO₄, 0.6 g of CaCl₂, 5 mg of FeSO₄. 7H₂O, 1.6 mg of MnSO₄, 1.4 mg of ZnSO₄7H₂O, 2 mg of CoCl2, and 10 g crab shell chitin in 1 liter of water.

Medium D contains the same nutrients as in medium C, but 10 g of cellulose was used instead of 10 g of chitin.

Medium E contains the same nutrients as in medium A, but 5 g of crab shell chitin and 5 g of cellulose were used instead of 10 g of chitin.

Medium F contains the same nutrients as medium C, but 5 g of crab shell chitin and 5 g of cellulose was used instead of 10 g of chitin.

Medium G contains 12.5 g of KH2PO4, 5 g of KNO3, 2.5 g of (NH₄)₂SO₄, 1.6 g of MgSO₄, 0.3 g of CaCl₂, 20 mg of FeCl₃, 2.5 mg of FeSO₄.7H2O, 2.5 mg of ZnSO₄, 7H₂O, 0.2 mg of CuSO₄5H₂O, 1 mg of MnSO₄, 0.06 mg of (NH₄)₆Mo₇O₂₄4H2O, 1 mg of CoCl₂, 2.5 g of lactose, 0.5 g of glucose, 0.25 g of sucrose, and 10 g of crab shell chitin in 1 liter of water.

Medium H contains the same nutrients as in medium G, but 10 g of cellulose was used instead of 10 g chitin in 1 liter of water.

The media were adjusted to pH 6.0 and sterilized by autoclaving in Erlenmeyer flasks (100 ml of medium in 250-ml flasks). Trace elements, especially FeCl₃ and FeSO₄, were sterilized separately and added immediately before use because some of these elements precipitate with time when mixed together. Crab shell chitin or cellulose was added separately into each flask before autoclaving. Each flask was inoculated with a spore suspension that contained approximately 5×10⁶ conidia ml⁻¹ and placed on a rotary shaker at 150 rpm at 25° C. for 5 to 6 days. The culture medium containing the enzymes of interest was separated from biomass by centrifugation at 8,000 rpm for 10 min. Residual particulates were removed by filtration through a glass fiber filter (type A/E, Gelman Sciences). The culture filtrates were then dialyzed using 12,000 m.w. cutoff standard cellulose dialysis tubing (Spectropor™ membrane tubing, Scientific Products West Chester, Pa.) against deionized water for 3 days. The deionized water was replaced every 4 hrs. For the enzyme solutions with high cellulase activities, the tubes were changed daily to avoid leakage due to action of cellulase on the cellulose membrane. After dialysis, the enzyme solutions were filter sterilized and kept at 4° C. until use.

Glucosaminidase and chitobiosidase by a modification of the procedures of Tronsmo et al., “Detection and Quantification of N-acetyl-β-D-Glucosaminidase, Chitobiosidase and Endochitinase in Solutions and on Gels,” Anal. Biochem. 208:74-79 (1993), which is hereby incorporated by reference in entirety. Briefly, assays measured the release of nitrophenol from p-nitrophenol-β-D-N-acetylglucosaminide or from p-nitrophenol-β-D-N,N′-diacetylchitobiose (both from Sigma St. Louis, Mo.) at pH 5.0 in microtiter plates. The yellow color of p-nitrophenol was read at 410 nm in a microtiter plate reader. Activity of the enzymes were expressed as nmoles of p-nitrophenol released per second per milliliter of culture filtrates. Endochitinase activity was measured by the reduction of turbidity of a suspension of colloidal chitin (Tronsmo et al., “Detection and Quantification of N-acetyl-β-D-Glucosaminidase, Chitobiosidase and Endochitinase in Solutions and on Gels,” Anal. Biochem. 208:74-79 (1993), which is hereby incorporated by reference in its entirety.). A suspension containing 1% (w/v) of moist colloidal chitin, purified as described by Vessey et al., “Autolysis and Chitinase Production in Cultures of Verticillium albo-atrum,” Trans Br. Mycol. Soc. 60:133-143 (1973), which is hereby incorporated by reference in its entirety, was prepared in 100 mM of acetate buffer, pH 5.0, and autoclaved. A mixture consisting of 125 μl each of the chitin suspension and the enzyme solution to be tested in a microplate well was prepared and incubated for 24 hr at 30 C. Subsequently, the mixture was read at 510 nm. Chitinase activity was calculated as the percentage of reduction in turbidity relative to that of a similar suspension that contained water rather than enzyme solution. One enzyme unit was defined as the amount of enzyme required to reduce the turbidity of a chitin suspension by 5% ((Tronsmo et al., “Detection and Quantification of N-acetyl-β-D-Glucosaminidase, Chitobiosidase and Endochitinase in Solutions and on Gels,” Anal. Biochem. 208:74-79 (1993), which is hereby incorporated by reference in its entirety.). Native polyacrylamide gel electrophoresis was used to identify specific protein and activity bands using methods noted in Example 2.

Antifungal activity of various samples was identified using assays in microplate wells. Each well contained 50 μl of PDB (potato dextrose broth, Difco), 50 μl of a conidial suspension (10⁵-10⁶ conidia ml⁻¹) of the test fungus Penicillium digitatum, and 50 μl of an enzyme solution (culture filtrates prepared as described above). Sterile water was added to bring the enzyme solution to a final volume of 50 μl when less than 50 μl 1 of enzyme solution was used for the assay. In the controls, sterile water was used in the place of enzyme solutions. The microplates were incubated without shaking for 12-14 hr at 25° C. After incubation, observations were taken directly from the microplate wells under a Nikon Diaphot brightfield microscope using a Pulnix TM-745 CCD video camera. The image analyzer used was an Image1/AT from Universal Imaging. However, microscope slides were also prepared for observation at higher levels of magnification. Because germ tubes tend to attach to the surface of the microplate well, a sterile tooth pick was used to scrape and suspend the germ tubes in solution before making the slide. Abnormal mycelial growth and morphological anomalies such as branching, bursting, appearance of necrotic zones, and lysis of the hyphal tips were recorded and photographed. Each experiment was repeated on two separate days and contained two treatment replicates each time.

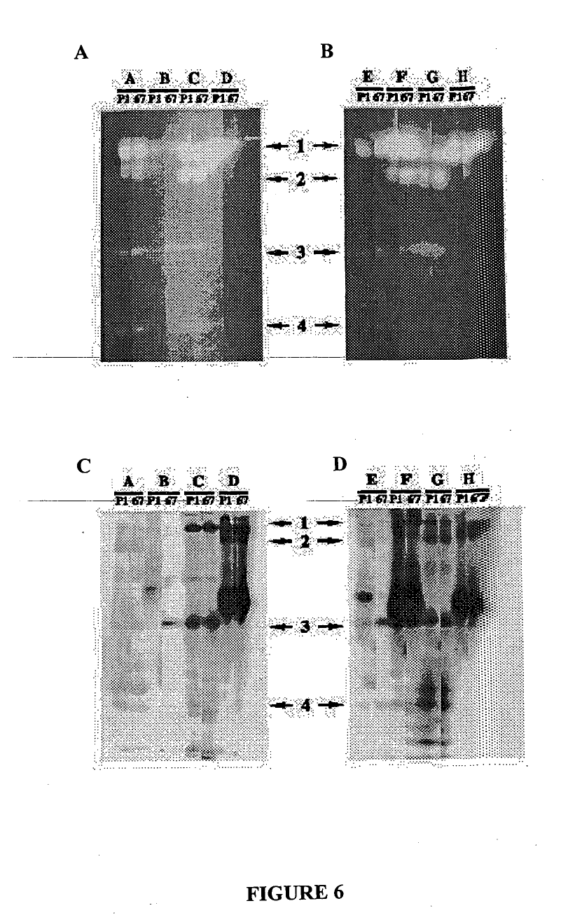

The intensity and pattern of activity bands varied depending on the strain and the medium. Strain 95467 showed an activity banding pattern for endochitinase similar to the wild type P1 in all media tested. The other chitinolytic enzymes (native to T. reesei) produced by 95467 banded at a location similar to those of P1, as shown in FIGS. 6A-D.

However, the levels of expression of specific enzymes were media-specific. When strain 95467 was grown in media B and E, it produced a relatively high level of almost pure endochitinase as shown in FIG. 6, plus some proteins that corresponded to the nahases. The intensity and banding patterns of the protein profiles of this strain were similar to those of the wild type P1 in many other growth media tested.

These data demonstrate that adjustments in the culture medium composition can dramatically affect the enzyme profiles of strain 95467. The high level of production of transgenic endochitinase with a smaller amount of nahases gives a mixture that can be highly efficient in destruction of fungal cells, as predicted from past work (U.S. Pat. No. 5,474,926 to Harman et al., which is hereby incorporated by reference in its entirety), since nahases and endochitinase are synergistic in their antifungal activity. In Table 1, below, the abilities of cultures of P1 and 95467 to prevent spore germination of the green mold pathogen of citrus, Penicillium digitatum, of the native strain (P1) are compared when both were grown in the either medium B or G.

TABLE 1
Antifungal activities of different culture filtrates against
Penicillium digilatum

| | | Protein | |
|---|---|---|---|
| Growth medium | | concentration* | Percent |
| Germination # | Strain | mg ml⁻¹ | % |
| B | P1 | 2.2 | 0 |
| | 67 | 1.0 | 0 |
| G | P1 | 5.2 | 0 |
| (spores destroyed) | | | |
| | 67 | 5.8 | 0 |
| (spores destroyed) | | | |
| Untreated control | — | — | 95-100 |

*Protein concentration in dialyzed culture filtrates
# Antifungal effect as indicated by reduction in spore germination when 50 μl of culture filtrate was assayed in a medium containing 50 μl of PDB and 50 μl of a P. digitatum spore suspension in water.

These data demonstrate that either the native or transgenic strain was effective in destruction of P. digitatum conidia even at the modest level of protein production attained. In actuality, culture filtrates from the transgenic strain were more effective than P1; in media G; even 5 μl of culture filtrate were highly effective.

Further, from these data, the concept stated earlier that nitrogen starvation, as induced by nitrate, can specifically enhance heterologous enzyme production. The cbh1 promoter is very highly induced in the presence of its inducer, cellulose (Mäntyläet al., “Industrial Mutants and Recombinant Strains of Trichoderma reesei,” in Harman and Kubicek (eds.): Trichoderma and Gliocladium, Vol. 2. Taylor and Francis, London, pp. 291-309 (1998), which is hereby incorporated by reference in its entirety). However, if the promoter is sufficiently effective, the resources of the producing organism will be primarily diverted to production of the enzyme of interest and other proteins will largely be limited. In the present example, the majority of the protein from strain 95467 was in the form of endochitinase, while in the wild strain P1 from which it was derived, most of the protein was other than endochitinase, including some proteins without chitinolytic activity, shown in FIG. 6.

In practice, stable antifungal enzymes may be useful for application to control pathogenic fungi in agricultural and other uses. If so, large-scale manufacturing processes are required, as has been done for other commercially useful enzymes, for instance, cellulases from Trichoderma spp. Production of EIMEs from Trichoderma and other organisms can be increased by modifying the growth conditions for enhanced induction, or through gene manipulation for overexpression of the enzymes of interest (Margolles-Clark et al., “Improved Production of Trichoderma harzianum Endochitinase by Expression in Trichoderma reesei,” Appl. Environ. Microbiol. 62:2145-2151 (1996), which is hereby incorporated by reference in its entirety) demonstrated that the endochitinase gene (ech42) from T. harzianum can be overexpressed in T. reesei about 20-fold with the cbh1 promoter of T reesei. A fivefold increase in the production of this enzyme was observed when this gene was expressed in T. harzianum downstream of the cbh1 promoter of T. reesei (Margolles-Clark et al.). Characterization of strain VTT-D-95467 prepared with this insertion and the antifungal enzymes produced by it provide information on the mechanisms involved in the antagonistic action of Trichoderma and factors affecting this complex biological process. Such strains may also be useful in development of a strategy for mass production of antifungal enzymes for biocontrol and other purposes.

### Example 8

### Synergism of Fungal and Bacterial Extracellular Compositions in N-Acetylglucosamine Production

Enzyme from T. atroviride strain P1 was produced in medium G as described in Example 7. This enzyme was used singly or in combination with an enzyme from the bacterium Streptomyces albidoflavus. The enzyme from this source was prepared as described in U.S. Pat. No. 6,069,299 to Broadway et al., and in Broadway et al., “Partial Characterization of Chitinolytic Enzymes from Streptomyces albidoflavus,” Lett. Appl. Microbiol. 20:271-276 (1995), which are hereby incorporated by reference in their entirety. The goal of the work was to release monomeric N-acetylglucosamine, or its polymers from crab shell chitin (“crab”) (Sigma, St. Louis, Mo.), chitin from langoustino shells (“Lang.”) or hydrated and swollen colloidal chitin (“CC”) from the same source. The colloidal chitin was prepared as described earlier (Vessey et al., “Autolysis and Chitinase Production in Cultures of Verticillium albo-atrum,” Trans Br. Mycol. Soc. 60:133-143 (1973), which is hereby incorporated by reference in its entirety). Results of digestion studies on the three chitin-containing source materials with enzyme from T. atroviride strain P1 and Streptomyces albidoflavzis are shown below in Table 2 and Table 3, respectively. Table 4 shows results of digestion under same conditions using a (1:1) mixture of T. atroviride strain P1 and Streptomyces albidoflavusextracellular enzymes, at a pH of 5.0. Data shown is from the first of two replicate experiments. Numbers presented in each column are g N-acetylglucosamine released per liter of reaction mixture, while the final percentages refer to the percentage that possibly could have been released based upon an estimate of the total amount of N-acetylglucosamine contained within chitin in the sample.

TABLE 2
Enzyme from T. atroviride strain P1
1^{st} digestion

| hour | crab | Lang. | CC |
|---|---|---|---|
| 0 | 0 | 0 | 0 |
| 3 | 0.12 | 0 | 0.6 |
| 24 | 0.61 | 2.8 | 5.2 |
| 48 | 0.30 | 3.2 | 10.5 |
| Eff. | 2% | 15% | 87% |

TABLE 3
Enzyme from Streptomyces albidoflavus
1^{st} digestion

| hour | SC | LG | CC |
|---|---|---|---|
| 0 | 0 | 0 | 0 |
| 3 | 0 | 0 | 0 |
| 24 | 0 | 0.8 | 0.5 |
| 48 | 0 | 1.4 | 2.3 |
| Eff. | 0 | 7% | 19% |

TABLE 4
(1:1) mix of enzyme from S. albidoflavus and T atroviride
1^{st} digestion

| hour | SC | LG | CC |
|---|---|---|---|
| 0 | 0 | 0 | 0.1 |
| 3 | 0.26 | 1.0 | 2.5 |
| 24 | 0.05 | 3.7 | 9.7 |
| 48 | 1.05 | 6.7 | 11.3 |
| Eff. | 8% | 33% | 95% |

As shown in Table 4, a mixture of enzymes was more effective than either one singly, and were, in fact, synergistic in their ability to degrade various forms of chitin to N-acetylglucosaminidase. To assess synergy, Limpel's formula as modified by Richer (Richer, “Synergism -a Patent View” Pestic. Sci. 19:309-315 (1987), which is hereby incorporated by reference in its entirety) was used. It states Eₑ=(X+Y)−(XY/100) where Eₑ is the expected combined effect; X is the expected effect when compound A is used alone and Y is the expected effect when compound B is used alone. If the observed effect (Eₒ) is greater than the expected effect (Eₑ), then synergism has been demonstrated. This synergy is readily evident: after 24 hr for colloidal, the values for X and Y are 5.2 and 0.5 mg for T. atroviride and S. albidoflavus enzymes, respectively, and Ee is calculated to be 5.4 mg, which is less than the observed value of 9.7 mg, thus demonstrating synergy. After 48 hr, the reaction with both T. atroviride and the combination of enzymes was approaching completion so differences were less obvious. Similar results can be obtained for the other two substrates, although for crab chitin, 48 hr values must be used given the relatively difficulty of degrading this substrate. Similar results with these enzyme preparations also have been obtained for degradation of fungal cell walls.

The economical production of N-acetylglucosamine (Nag) and other products from natural sources depends upon enzyme sources that are relatively inexpensive. It may be difficult to prepare enzymes inexpensively enough for commercial practice if the enzymes can be used only once. However, economic viability can be dramatically increased if enzymes can be reused. Therefore, the reaction mixture from the first digestion reaction was recovered after the 48 hr digestion of langoustino shells by enzyme from T. atroviride. This consisted of an oatmeal-like paste consisting of debris from the digestion plus enzyme in solution and an additional relatively large quantity of enzyme that remained bound to the undigested shell remnants.

The particulate was separated from the supernatant. A substantial amount of enzyme activity was present in both fractions. Additional chitin and a small amount (about 10% of the original activity) of fresh enzyme, were added to the recovered fractions, with no separation of Nag or other materials. These “residual” enzymes were effective in both the supernatant and particulate fractions, and were about 70% as effective in release of Nag as the original enzyme sample even though 90% of the enzyme had been used once, and it was diluted by separation into two samples. This data suggests that the enzyme can be recycled and reused a number of times, thereby making the process commercially. economical.

### Example 9

### Digestion Reaction at Alkaline pH

In the preceding example, reactions all were carried out a pH 5. Digestion reactions were carried out under conditions similar to those described in Example 8, at pH 9, with the enzyme from S. albidoflavus. Results from attempts to detect N-acetylglucosamine are shown in Table 5.

TABLE 5
1^{st} digestion

| hour | SC | LG | CC |
|---|---|---|---|
| 0 | 0 | 0 | 0 |
| 3 | 0 | 0 | 0 |
| 24 | 0 | 0 | 0 |
| 48 | 0 | 0 | 0 |
| Eff. | 0 | 0 | 0 |

What is claimed:

1. A method of increasing expression of native extracellular inducible microbial enzymes from Trichoderma or Gliocladium species, said method comprising:
culturing Trichoderma or Gliocladium species in a medium containing material sufficient to induce expression of native extracellular inducible microbial enzymes, low levels of carbohydrates, and/or low levels of reduced nitrogen compounds under conditions effective to increase expression of native fungal extracellular inducible microbial enzymes.

2. A method according to claim 1, wherein the medium further comprises a level of chitin containing material sufficient to induce expression of native extracellular inducible microbial enzymes.

3. A method according to claim 1, wherein said culturing is carried out with a low level of carbohydrates.

4. A method according to claim 1, wherein said culturing is carried out with a low level of reduced nitrogen compounds.

5. A method according to claim 1, wherein the medium further comprises a level of chitin containing material sufficient to induce expression of native extracellular inducible microbial enzymes and a low level of carbohydrates.

6. A method according to claim 1, wherein the medium further comprises a level of chitin containing material sufficient to induce expression of native extracellular inducible microbial enzymes and a low level of reduced nitrogen compounds.

7. A method according to claim 1, wherein said culturing is carried out with a low level of carbohydrates and a low level of reduced nitrogen compounds.

8. A method according to claim 1, wherein the carbohydrates are present in the medium at a level of 5 to 50 mM.

9. A method according to claim 1, wherein the reduced nitrogen compound is present in the medium at a level of 3 to 40 mM.

10. A method according to claim 1, wherein the carbohydrates are selected from the group consisting of hexoses, pentoses, sugar alcohols, disaccharides, amino sugars, easily degraded α-linked polymers, oligomers thereof, and mixtures thereof.

11. A method according to claim 1, wherein the carbohydrates are selected from the group consisting of glucose, galactose, mannose, fructose, xylose, ribose, mannitol, sorbitol, glucosamine, galactosamine, starch, sucrose, maltose, and mixtures thereof.

12. A method according to claim 1, wherein the reduced nitrogen compound is selected from the group consisting of ammonium salts, glutamine, urea, amino acids and mixtures thereof.

13. A method according to claim 1, wherein the extracellular inducible enzymes are selected from a group consisting of enzymes that degrade chitin, β-1,3 glucans, cellulose, hemicellulose, phytic acid, and proteins.

14. A method according to claim 13, wherein the extracellular inducible enzymes degrade chitin.

15. A method according to claim 13, wherein the extracellular inducible enzymes degrade β-1,3 glucans.

16. A method according to claim 13, wherein the extracellular inducible enzymes degrade cellulose.

17. A method according to claim 13, wherein the extracellular inducible enzymes degrade hemicellulose.

18. A method according to claim 13, wherein the extracellular inducible enzymes degrade phytic acid.

19. A method, in a culture of fungi, of repressing expression of native proteins and enhancing expression of proteins encoded by transgenes, said method comprising:
culturing fungi, which have been transformed with at least one transgene controlled by a promoter, in a medium containing an inducer of the promoter, high levels of carbohydrates, and/or high levels of reduced nitrogen compounds under conditions effective to repress expression of native proteins and to enhance expression-of proteins encoded by the at least one transgene.

20. A method according to claim 19, wherein said culturing is carried out with an inducer of the promoter.

21. A method according to claim 19, wherein said culturing is carried out with a high level of carbohydrates.

22. A method according to claim 19, wherein said culturing is carried out with a high level of reduced nitrogen compounds.

23. A method according to claim 19, wherein said culturing is carried out with an inducer of the promoter and a high level of carbohydrates.

24. A method according to claim 19, wherein said culturing is carried out with an inducer of the promoter and a high level of reduced nitrogen compounds.

25. A method according to claim 19, wherein said culturing is carried out with a high level of carbohydrates and a high level of reduced nitrogen compounds.

26. A method according to claim 19, wherein the carbohydrates are present in the medium at a level of 100 mM to 500 mM.

27. A method according to claim 19, wherein the reduced nitrogen compound is present in the medium at a level of 35 mM to 500 mM.

28. A method according to claim 19, wherein the carbohydrates are selected from the group consisting of hexoses, pentoses, sugar alcohols, disaccharides, amino sugars, easily degraded a-linked polymers, oligomers thereof, and mixtures thereof.

29. A method according to claim 19, wherein the carbohydrates are selected from the group consisting of glucose, galactose, mannose, fructose, xylose, ribose, mannitol, sorbitol, glucosamine, galactosamine, starch, sucrose, maltose, and mixtures thereof.

30. A method according to claim 19, wherein the reduced nitrogen compound is selected from the group consisting of ammonium salts, glutamine, urea, amino acids, and mixtures thereof.

31. A composition comprising:
a fungal source of a extracellular inducible microbial enzyme and
a bacterial source of a extracellular inducible microbial enzyme.

32. A composition according to claim 31, wherein the fungal source is a filamentous fungus or a yeast.

33. A composition according to claim 32, wherein the fungal source is Trichoderma spp., Rhizopus spp., Aphanocladium spp., Coccidioides spp., Aspergillus spp., Hyprocrea spp., Candida spp., or mixtures thereof.

34. A composition according to claim 31, wherein the bacterial source is Aeromonas spp., Streptomyces spp., Serratia spp., Bacillus spp., Chromobacter spp., Vibrio spp., Pseudomonas spp., Pyrococcus spp., Aeromonas spp., or mixtures thereof.

35. A method of releasing N-acetylglucosamine from a chitinous source comprising:
treating a chitinous source with a composition comprising:
a fungal source of a extracellular inducible microbial enzyme and
a bacterial source of a extracellular inducible microbial enzyme, said treating being carried out under conditions effective to release N-acetylglucosamine from the chitinous source.

36. A method according to claim 35, wherein the fungal source is a filamentous fungus or a yeast.

37. A method according to claim 36, wherein the fungal source is Trichoderma spp., Rhizopus spp., Aphanocladium spp., Coccidioides spp., Aspergillus spp., Hyprocrea spp., Candida spp., or mixtures thereof.

38. A method according to claim 35, wherein the bacterial source is Aeromonas spp., Streptomyces spp., Serratia spp., Bacillus spp., Chromobacter spp., Vibrio spp., Pseudomonas spp., Pyrococcus spp., or mixtures thereof.

39. A method according to claim 35, wherein said treating is carried out under alkaline pH conditions effective to produce an oligomer of N-acetylglucosamine.

40. A method of repeatedly degrading different chitinous substrates comprising:
(a) treating a first chitinous substrate with a composition comprising:
a fungal source of a extracellular inducible microbial enzyme and/or
a bacterial source of a extracellular inducible microbial enzyme, under conditions effective to produce chitin;
(b) terminating step (a);
(c) treating a second chitinous substrate with the same composition used to carry out step (a) under conditions effective to produce chitin.

41. A method according to claim 40, wherein, after termination of step (c), repeating step (c) with the same compositions used to carry out step (c).

42. A method according to claim 40, wherein the fungal source is a filamentous fungus or a yeast.

43. A method according to claim 42, wherein the fungal source is Trichoderma spp., Rhizopus spp., Aphanocladium spp., Coccidioides spp., Aspergillus spp., Hyprocrea spp., Candida spp., or mixtures thereof.

44. A method according to claim 40, wherein the bacterial source is Aeromonas spp., Streptomyces spp., Serratia spp., Bacillus spp., Chromobacter spp., Vibrio spp., Pseudomonas spp., Pyrococcus spp., or mixtures thereof.

45. A method of increasing expression of native extracellular inducible microbial enzymes from Trichoderma or Gliocladium species, said method comprising:
culturing Trichoderma or Gliocladizim species in a medium containing material sufficient to induce expression of native extracellular inducible microbial enzymes, a carbon source not repressive of expression of fungal extracellular inducible microbial enzymes, and/or low levels of reduced nitrogen compounds under conditions effective to increase expression of native fungal cell extracellular inducible microbial enzymes.

46. A method according to claim 45, wherein the medium further comprises a level of chitin containing material sufficient to induce expression of native cell extracellular inducible microbial enzymes.

47. A method according to claim 45, wherein said culturing is carried out with a carbon source not repressive to fungal extracellular inducible microbial enzymes.

48. A method according to claim 45, wherein said culturing is carried out with a low level of reduced nitrogen compounds.

49. A method according to claim 45, wherein the medium further comprises a level of chitin containing material sufficient to induce expression of native extracellular inducible microbial enzymes and a carbon source not repressive to fungal extracellular inducible microbial enzymes.

50. A method according to claim 45, wherein the medium further comprises a level of chitin containing material sufficient to induce expression of native extracellular inducible microbial enzymes and a low level of reduced nitrogen compound.

51. A method according to claim 45, wherein said culturing is carried out with a carbon source not repressive to expression of fungal extracellular inducible microbial enzymes and a low level of reduced nitrogen compound.

52. A method according to claim 45, wherein the reduced nitrogen compound is present in the medium at a level of 2 to 30 mM.

53. A method according to claim 45, wherein the nitrogen compound is selected from the group consisting of ammonium salts, glutamine, urea, amino acids and mixtures thereof.

54. A method of enhancing purity of a heterologous recombinant protein expressed in a culture of fungi, said method comprising:
culturing the recombinant fungi in a medium containing nitrate as the medium's sole nitrogen source under conditions effective to induce expression of the heterologous protein while reducing expression of other proteins.
